(19) United States
(12) Patent Application Publication
(10) Pub. No.: US 2024/0050722 A1
(43) Pub. Date: Feb. 15, 2024

(54) LAVAGE ARRANGEMENT

(71) Applicant: H & B Electronic GmbH & Co. KG, Deckenpfronn (DE)

(72) Inventors: Tobias MORLOK, Mötzingen (DE); Jan BAYERBACH, Calw (DE); Thomas WEINMANN, Neuweiler (DE)

(73) Assignee: H & B Electronic GmbH & Co. KG, Deckenpfronn (DE)

(21) Appl. No.: 18/230,377

(22) Filed: Aug. 4, 2023

(30) Foreign Application Priority Data
Aug. 9, 2022 (DE) 102022120056.6

(51) Int. Cl. A61M 35/00; A61M 3/02

(52) U.S. Cl. CPC A61M 35/00; A61M 3/0258; A61M 2202/0468; A61M 2205/103; A61M 2205/8206

(57) ABSTRACT

A lavage arrangement has an applicator housing which has a handle and an application opening for dispensing a rinsing liquid and encloses a drive receptacle which is accessible via a housing opening. The lavage arrangement has a liquid line by means of which the application opening can be connected to a reservoir of the rinsing liquid. The lavage arrangement has a drive unit of a pump that can be inserted into the drive receptacle, for conveying the rinsing liquid from the reservoir to the application opening, the drive unit having a motor, a power supply device and a pump drive shaft which can be driven by the motor. A rotor which can be driven by means of the pump drive shaft is provided, on which at least one displacement element is held and which, together with a deformable section of the liquid line forms the pump in the form of a peristaltic pump.

The invention relates to a lavage arrangement for rinsing out and cleaning body parts or wounds by means of spray or flushing during an operation, such as when inserting artificial joints or prostheses or during wound debridement. For this purpose, the lavage arrangement has an applicator housing that forms a handle and an application opening for dispensing a rinsing liquid, which is formed in particular by water or an aqueous solution containing medication and/or cleaning substances, such as an NaCl solution. In addition, the applicator housing encloses a drive receptacle that is accessible via a housing opening. Furthermore, the lavage arrangement has a liquid line, by means of which the application opening is or can be connected to a supply of a rinsing liquid. In addition, the lavage arrangement has a drive unit of a pump that can be inserted into the drive receptacle and is used to convey the rinsing liquid from the supply to the application opening. For this purpose, the drive unit has a motor, a power supply device and a pump drive shaft that can be driven by the motor.

Such a lavage arrangement enables gentle and thorough rinsing of the affected operation areas, in particular by means of recurring flushing. In this way, bone, tissue or material residues and other contamination that occur during an operation can be removed, which in turn can prevent infections and accelerate healing processes.

A lavage arrangement is known from EP3706823 B1, which has a drive unit with a motor and a battery. The drive unit can be inserted into a housing in an exchangeable manner and has a shaft with a drive gear wheel. In the inserted state, the drive gear meshes with a pump gear of a piston pump, which is held on the housing.

Due to the exchangeable reception of the drive unit in the housing, the latter can be used as a disposable product and, after use, can be disposed of for hygienic reasons, while the drive unit can be reused together with another, unused housing.

A disadvantage of the known lavage arrangement with a reusable drive unit is that the cost and material expenditure for the housing, which is designed as a disposable part, with the parts attached thereto that are required for the pump function, are relatively high.

The object of the invention is to avoid the disadvantages mentioned in a generic lavage arrangement and to further reduce the cost and the consumption of resources.

This object is achieved by a lavage assembly having the features of claim 1. In this case, the lavage arrangement has a rotor which can be driven by the pump drive shaft and on which at least one displacement element is held, the rotor together with a deformable section of the liquid line forming the pump, specifically in the form of a peristaltic pump. This use of a peristaltic pump for conveying the rinsing liquid can significantly reduce the manufacturing cost both of the pump and of the lavage arrangement overall. In addition, the parts required for the pump function on the housing side can be reduced to a minimum. Thus, both the cost and the resources required for repeated use of the lavage arrangement can be significantly reduced overall.

The rotor, together with the at least one displacement element, is preferably held on the drive unit and can be inserted into the drive receptacle interchangeably with said drive unit as a unit. In this way, the parts required for the pump function can be reused to a very large extent, while the parts required for the pump function, which are disposed of after use as disposable elements, are reduced to a minimum.

The deformable section of the liquid line is advantageously held on the applicator housing and the rotor can be placed against the deformable section when the drive unit is inserted into the drive receptacle. As a result, at least the deformable section of the liquid line can be used as a disposable element of the pump, which is also disposed of when the application housing is disposed of. In this way, it can be ensured that when the drive unit is reused with a still unused applicator housing, there is also an unused, deformable section of the liquid line. In this way, when the drive unit is reused with a new applicator housing with a deformable section, sterility can be guaranteed on the one hand and the occurrence of leaks on the peristaltic pump as a result of wear can be avoided.

In a particularly advantageous embodiment, at least three displacement elements are held on the rotor. As a result, the deformable section of the liquid line can be repeatedly compressed at relatively short intervals when the rotor is driven. In this way, recurring flushing is generated from the application opening at short time intervals.

In addition, it is favorable if the displacement elements have at least one roller. All displacement elements are preferably designed in the form of rollers. This enables the deformable section of the liquid line to be compressed evenly and in a manner that is gentle on the material. As an alternative to the roller, at least one displacement element can be formed as a sliding element held immovably on the rotor in order to reduce the maintenance effort, for example.

It is favorable here if the deformable section of the liquid line is formed by a tube with a rounded cross section, such as in particular a circular or elliptical cross section, which can be placed against a correspondingly rounded concave contact area of the applicator housing. As a result, a uniform, planar contact of the liquid line on the applicator housing is ensured during the compression of the deformable section. In this way, damage to the liquid line can be avoided during the pumping process.

Advantageously, the at least one displacement element of the rotor has a contact surface that can be placed against the deformable section and has a rounded transverse profile formed transversely to a conveying direction. As a result, an essentially complete compression of the deformable section is possible, as a result of which a high pump capacity and strong flushing can be generated.

It is advantageous if the housing opening can be closed by a closing element that is movably held on the rest of the applicator housing. In this way, the closing element can be held captive on the remainder of the applicator housing in order to be able to handle the latter both before and during use of the lavage arrangement and during disposal as a one-piece disposable element.

In this case, the closing element is advantageously formed by a hinged cover, as a result of which the application opening of the applicator housing can be opened or closed particularly easily and quickly in order to be able to remove or install the drive unit.

In addition, it is advantageous if the applicator housing is made of a single plastic material together with the closing element, in order to enable easy recycling of the disposable part.

Advantageously, a rinsing attachment, such as a funnel; a tube or a hollow needle can be attached interchangeably in order to be able to generate a spray or rinsing jet suitable for the respective application. In addition, a suction opening can also be provided on the rinsing attachment, via which the rinsing liquid can be transported away after rinsing, together with the particles that have been rinsed off.

The reservoir of the rinsing liquid is preferably formed by a separate container that can be connected to the applicator housing via a supply section of the liquid line. As a result, when using the lavage arrangement, the reservoir does not have to be carried along on the applicator housing and can essentially be dimensioned as desired.

The motor and the power supply device are preferably received in a protected manner in a drive housing of the drive unit. For this purpose, the drive housing is designed in particular to be watertight in order to avoid damage to the motor and the power supply device if the drive unit is cleaned or disinfected or if rinsing liquid escapes. This allows multiple reuse of the drive unit.

Furthermore, the power supply device is advantageously formed by a rechargeable battery in order to be able to provide the power supply to the drive unit on a recurring basis in a simple manner. As an alternative, the power supply device can also be formed by a power cable connection in order to enable a substantially unlimited power supply.

It should be noted that all features of the subject according to the invention described above can be exchanged or combined with one another, provided that an exchange or a combination thereof is not ruled out for technical reasons.

An exemplary embodiment of the invention is shown in the figures. In the figures:

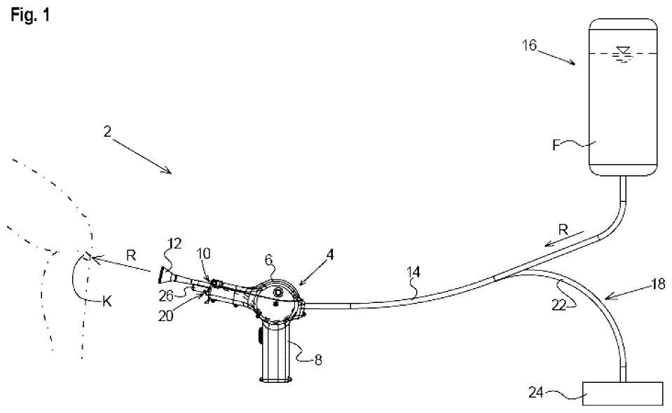

FIG. 1 shows a view of a lavage arrangement according to the invention,

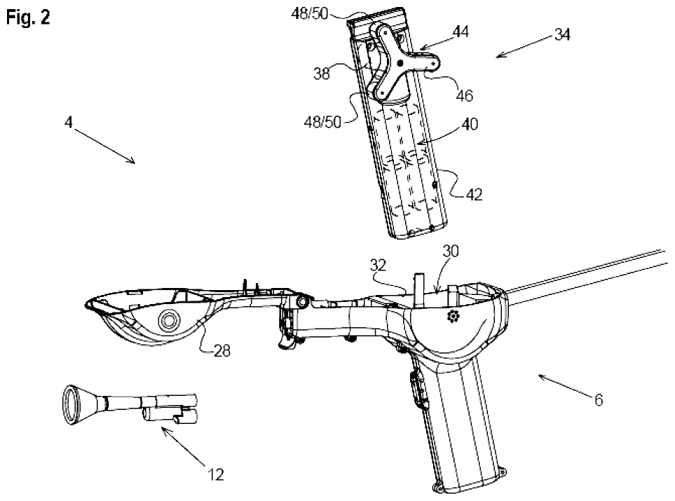

FIG. 2 shows an exploded view of an applicator of the lavage arrangement according to FIG. 1,

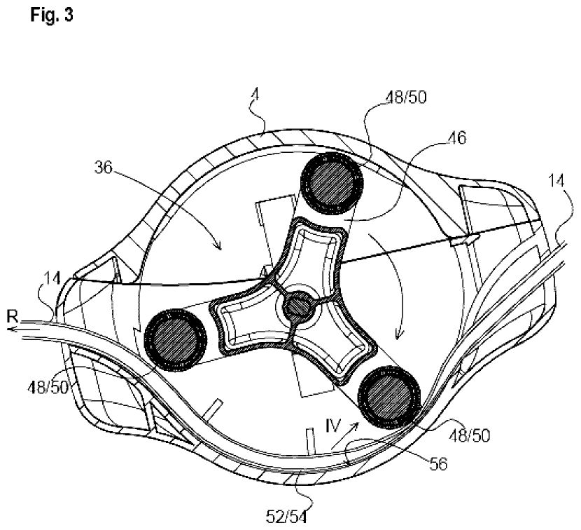

FIG. 3 shows a section through a peristaltic pump of the applicator according to FIG. 2 and

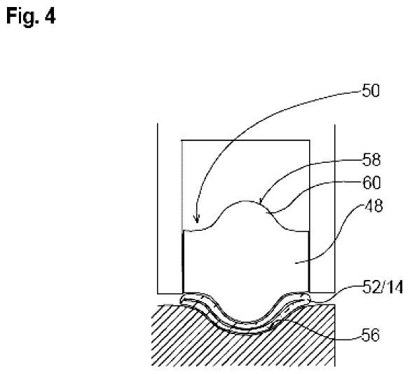

FIG. 4 shows a partially sectioned plan view of a displacement element of the peristaltic pump in direction IV from FIG. 3.

FIG. 1 shows a lavage arrangement 2 for rinsing out and cleaning a treated part of a body K in the form of a wound or a bone during an operation, such as in the case of an insertion of an artificial joint or a prosthesis. For this purpose, the lavage arrangement 2 has an applicator 4 with an applicator housing 6. Said applicator housing 6 forms a handle 8 and has an application opening 10 to which a rinsing attachment 12 can be attached in an exchangeable manner. Depending on the type of application, a rinsing attachment 12 in the form of a tube or hollow needle or, as shown, in the form of a funnel can be used.

Application opening 10 is connected to a separate reservoir 16 via a liquid line 14 that is partially routed within the applicator housing 6, in which reservoir 16 a rinsing liquid F that can be used for the rinsing process is stored.

Furthermore, lavage arrangement 2 preferably has a suction arrangement 18 with a suction connection 20 which can also be provided on application opening 10 and is connected to a suction hose 22. Suction hose 22 can be subjected to a negative pressure, for example by connecting it to a stationary negative pressure device 24. The stationary negative pressure device 24 can be formed, for example, by a surgical suction device of a hospital or a doctor's practice.

In addition to being connected to liquid line 14, funnel-shaped rinsing attachment 12 shown can also have a suction attachment 26 via which it can be connected to suction connection 20. Funnel-shaped rinsing attachment 12 can thus be placed on the relevant part of the body K during a rinsing procedure in order to be able to suck in dispensed rinsing liquid 16 within the funnel and to conduct it away via suction hose 22 together with the particles detached during rinsing.

As can be seen in particular from FIG. 2, applicator housing 6 has a movably held closing element 28 in the form of a hinged cover, which can be folded into an open position shown relative to the rest of applicator housing 6. In this open position, a drive receptacle 30 is accessible via a housing opening 32. Drive receptacle 30 serves to receive and fix a removable drive unit 34 for a pump 36 (according to FIG. 3). For this purpose, drive unit 34 has a motor 38 and a power supply device 40 in the form of a preferably rechargeable battery. As an alternative to using a battery, power supply device 40 can also be formed by a power cable connection. In any case, motor 38 and power supply device 40 are protected in a preferably watertight drive housing 42 that allows drive unit 34 to be cleaned and disinfected multiple times without damaging motor 38 and power supply device 40.

A pump drive shaft 44 that can be driven by motor 38 is provided on the outside of drive housing 42. A rotor 46 is held on said pump drive shaft 44, on which at least one displacement element 48, preferably at least three displacement elements 48 arranged circumferentially, are mounted. Displacement elements 48 are formed, for example, by rollers 50 rotatably mounted on rotor 46. As an alternative, displacement elements 48 can also be formed by sliding elements which are firmly connected to rotor 46 (not shown).

In any case, displacement elements 48 are used together with a deformable section 52 of liquid line 14, according to FIG. 3, to form pump 36 as a peristaltic pump. Deformable section 52 is formed by a tube 54 with a circular or elliptical cross section, which is routes or held on a concave contact area 56 on the inside of applicator housing 6.

Peristaltic pump 36 can be activated by rotor 46 driven by motor 38 in order to convey rinsing liquid F along conveying direction R from storage container 16 via liquid line 14 to applicator 4 and to deliver as recurring flushing at application opening 10 or at rinsing attachment 12 placed on it.

For this purpose, the deformable section 52 according to FIG. 3 is impinged successively and repeatedly by at least one displacement elements 48 upon rotational movement of rotor 46. Said displacement elements 48 compress the deformable section 52 together in each case and simultaneously move in the conveying direction R, as a result of which the rinsing liquid F contained in liquid line 14 is also transported in conveying direction R. Depending on whether displacement elements 48 are designed as rollers 50 or sliding elements, they roll or slide on the tube. In order to enable the most complete possible compression of deformable section 52, displacement elements 48 have a contact surface 58 with a transverse profile 58 that is designed to be essentially complementary to concave contact area 56, as shown in FIG. 4 by way of example for one of rollers 50.

After use of lavage arrangement 2 according to FIG. 1, drive unit 34, which during operation was received splash-proof in applicator housing 6, can be removed from drive receptacle 30 upon opening of closing element 28, and inserted in an unused applicator housing 6 for reuse.

Applicator housing 6 that has already been used, which typically has contamination on its outside as a result of flushing generated during operation, can, on the other hand, be disposed of as a disposable element for hygienic reasons. For easier disposal or for more efficient recycling, applicator housing 6 including closing element 28, are preferably made of a single plastic. A rinsing attachment 12 used in the application that has taken place and at least deformable section 52 of liquid line 14 can also be disposed of as further disposable elements.

Before the drive unit 34 is reused with the still unused new applicator housing 6, power supply device 40 formed by a battery can also be charged or replaced. In this way, when drive unit 34 is reused, the full performance of motor 38 and pump 36 and the lavage arrangement as a whole can be guaranteed

It should be noted that all elements and features of the various embodiments of the subject according to the invention described above can be exchanged or combined with one another, provided that an exchange or a combination thereof is not ruled out for technical reasons.

1. A lavage arrangement, comprising:
an applicator housing having a handle and an application opening for dispensing a rinsing liquid, the applicator housing enclosing a drive receptacle which is accessible via a housing opening;
a liquid line by means of which the application opening can be connected to a reservoir of the rinsing liquid; and
a drive unit of a pump that can be inserted into the drive receptacle for conveying the rinsing liquid from the reservoir to the application opening, the drive unit having a motor, a power supply device and a pump drive shaft which can be driven by the motor,
wherein a rotor which can be driven by the pump drive shaft is provided with at least one displacement element, the at least one displacement element, together with a deformable section of the liquid line, forming the pump in the form of a peristaltic pump, and wherein the rotor is held with the at least one displacement element on the drive unit and can be inserted interchangeably with the drive unit in the drive receptacle.

2. The lavage arrangement according to claim 1, wherein the liquid line is held on the applicator housing and the rotor can be placed against the deformable section when the drive unit is inserted into the drive receptacle.

3. The lavage arrangement according to claim 1, wherein at least three displacement elements are held on the rotor.

4. The lavage arrangement according to claim 1, wherein the displacement elements have at least one roller.

5. The lavage arrangement according to claim 1, wherein the deformable section of the liquid line is formed by a tube with a rounded cross section, and wherein the tube can be placed against a correspondingly rounded concave contact area of the applicator housing.

6. The lavage arrangement according to claim 5, wherein the at least one displacement element has a contact surface that can be placed against the deformable section and has a rounded, concave transverse profile formed transversely to a conveying direction.

7. The lavage arrangement according to claim 1, wherein the housing opening can be closed by a closing element held movably on a remainder of the applicator housing.

8. The lavage arrangement according to claim 7, wherein the closing element is formed by a hinged cover.

9. The lavage arrangement according to claim 7, wherein the applicator housing with the closing element is made of a single plastic material.

10. The lavage arrangement according to claim 1, wherein a rinsing attachment can be attached exchangeably to the application opening.

11. The lavage arrangement according to claim 1, wherein the reservoir of the rinsing liquid is formed by a separate container which can be connected to the applicator housing via the liquid line.

12. The lavage arrangement according to claim 1, wherein the motor and the power supply device are received in a protected manner in a drive housing of the drive unit.

13. The lavage arrangement according to claim 1, wherein the power supply device is formed by a rechargeable battery.

14. The lavage arrangement according to claim 2, wherein at least three displacement elements are held on the rotor.

15. The lavage arrangement according to claim 2, wherein the displacement elements have at least one roller.

16. The lavage arrangement according to claim 3, wherein the displacement elements have at least one roller.

17. The lavage arrangement according to claim 2, wherein the deformable section of the liquid line is formed by a tube with a rounded cross section, and wherein the tube can be placed against a correspondingly rounded concave contact area of the applicator housing.

18. The lavage arrangement according to claim 3, wherein the deformable section of the liquid line is formed by a tube with a rounded cross section, and wherein the tube can be placed against a correspondingly rounded concave contact area of the applicator housing.

19. The lavage arrangement according to claim 4, wherein the deformable section of the liquid line is formed by a tube with a rounded cross section, and wherein the tube can be placed against a correspondingly rounded concave contact area of the applicator housing.

20. The lavage arrangement according to claim 2, wherein the housing opening can be closed by a closing element held movably on a remainder of the applicator housing.
